(19) United States
(12) Patent Application Publication
(10) Pub. No.: US 2014/0058368 A1
(43) Pub. Date: Feb. 27, 2014

(54) FORWARD FIRING FLAT TIP SURGICAL LASER FIBER ASSEMBLY

(71) Applicant: Hogue Surgical, LLC, Maple Grove, MN (US)

(72) Inventor: Roger S. Hogue, St. Peter, MN (US)

(73) Assignee: Hogue Surgical, LLC, Maple Grove, MN (US)

(21) Appl. No.: 13/965,736

(22) Filed: Aug. 13, 2013

Related U.S. Application Data
(60) Provisional application No. 61/691,855, filed on Aug. 22, 2012.

(51) Int. Cl. A61B 18/22

(52) U.S. Cl. CPC A61B 18/22
USPC 606/16

(57) ABSTRACT

A forward firing optical fiber assembly includes an optical fiber and a capillary tube. The optical fiber includes a substantially flat end face at a distal end of the optical fiber that extends in a plane substantially perpendicular to a longitudinal axis of the optical fiber. The capillary tube includes a rounded distal tip, and the distal end of the optical fiber disposed within the capillary tube such that the end face of the optical fiber is disposed a distance from an interior distal end of the capillary tube. The forward firing optical fiber assembly includes a fusion region between the optical fiber and capillary tube configured to hermetically seal the end face within the capillary tube.

### CROSS-REFERENCE TO RELATED APPLICATIONS

This application claims the benefit of Application No. 61/691,855, filed Aug. 22, 2012, which is incorporated herein by reference in its entirety.

### TECHNICAL FIELD

The present disclosure relates to a device for delivering laser energy for localized applications. More particularly, the present disclosure relates to a forward firing flat tip surgical laser fiber assembly.

### BACKGROUND

Traditionally, a forward firing surgical laser fiber has features of having a flat bare tip, either mechanically polished or cleaved, with a fiber end face perimeter that is potentially sharp and can cut tissue during passage to a target site. Additionally, the discharged laser energy in a fiber delivery laser system has its highest density of energy, or fluence of laser energy for exposed tissue, at the distal fiber end face. At the distal fiber end face surface of the flat bare tip, where direct exposure to tissue occurs, high microthermal temperatures, tissue ablation, carbonization, and end face contamination may occur, thereby affecting laser energy transmission at the fiber end face. Additionally, the sharp edge perimeter of the flat bare tip silica fiber end face has the potential for causing unintended tissue trauma, laceration, and/or transection, potentially resulting in bleeding or hemorrhaging.

In some cases, it may be possible to eliminate the sharp circumferential edge by modifying the end face of the fiber to include a ball-tip or orb-tip configuration. This modification may be provided by, for example, melting the fiber tip with a laser, such as a CO₂ laser. However, modification of the fiber end face with a ball-tip configuration can result in a weakening of the fiber tip caused by the melting and narrowing of the silica. In addition, while this type of modification reduces sharpness of the fiber, the highest fluence is still at the convex surface of the fiber end face, which can result in thermal induced silica glazing, carbonization, tissue adherence to the silica with super heating and carbonization, and end face contamination.

### SUMMARY

In one aspect, the present disclosure relates to a forward firing optical fiber assembly including an optical fiber and a capillary tube. The optical fiber includes a substantially flat bare end face at a distal end of the optical fiber that extends in a plane substantially perpendicular to a longitudinal axis of the optical fiber. The capillary tube includes a rounded, convex distal tip, and the distal end of the optical fiber disposed within the capillary tube such that the substantially flat end face of the optical fiber is disposed a distance from an interior distal end of the capillary tube. The forward firing optical fiber assembly includes a one or more fusion regions between the optical fiber and capillary tube configured to hermetically seal the end face within the capillary tube.

In another aspect, the present disclosure relates to a surgical laser system comprising a laser source and an optical fiber assembly optically coupled to the laser source. The optical fiber assembly includes an optical fiber with a fused silica cladding and core having a substantially flat bare tip end face at a distal end of the optical fiber. The end face extends in a plane substantially perpendicular to a longitudinal axis of the optical fiber. The optical fiber assembly further includes a capillary tube having a rounded, convex distal tip. The distal end of the optical fiber is disposed within the capillary tube such that the substantially flat end face of the optical fiber is disposed a distance from an interior distal end of the capillary tube. The forward firing optical fiber assembly also includes one or more fusion regions between the fused silica cladding and core and capillary tube and is configured to hermetically seal the end face within the capillary tube. The optical fiber assembly is configured such that laser energy provided by the laser source to the optical fiber assembly is transmitted through the end face forward through an air chamber within the capillary tube, then through a distal end of the capillary tube.

In a further aspect, the present disclosure relates to a forward firing optical fiber assembly including an optical fiber, a capillary tube, and a fusion region. The optical fiber includes a buffer layer, one or more silica cladding layers, and a silica core. The buffer and cladding layers are removed at a distal end portion of the optical fiber to expose the fused silica cladding and core layers, and the silica bare tip includes a substantially flat end face at a distal end of the optical fiber. The flat silica end face extends in a plane substantially perpendicular to a longitudinal axis of the optical fiber. The capillary tube includes a rounded distal tip, and the distal end of the optical fiber disposed within the capillary tube such that the substantially flat end face of the optical fiber is disposed a distance from an interior distal end of the capillary tube. One or more fusion regions between the optical fiber and capillary tube are configured to hermetically seal the fused silica cladding and core layers within the capillary tube.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic representation of an embodiment of a forward-firing surgical laser fiber assembly including an optical fiber with a flat end face.

FIG. 2A is a side view of an optical fiber distal end portion with a flat end face disposed within a capillary tube.

FIG. 2B is a cross-sectional view of the optical fiber distal end portion shown in FIG. 2A.

FIG. 3 is a side view of the optical fiber distal end portion illustrating the connection at the transition section between the optical fiber buffer and the capillary tube.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

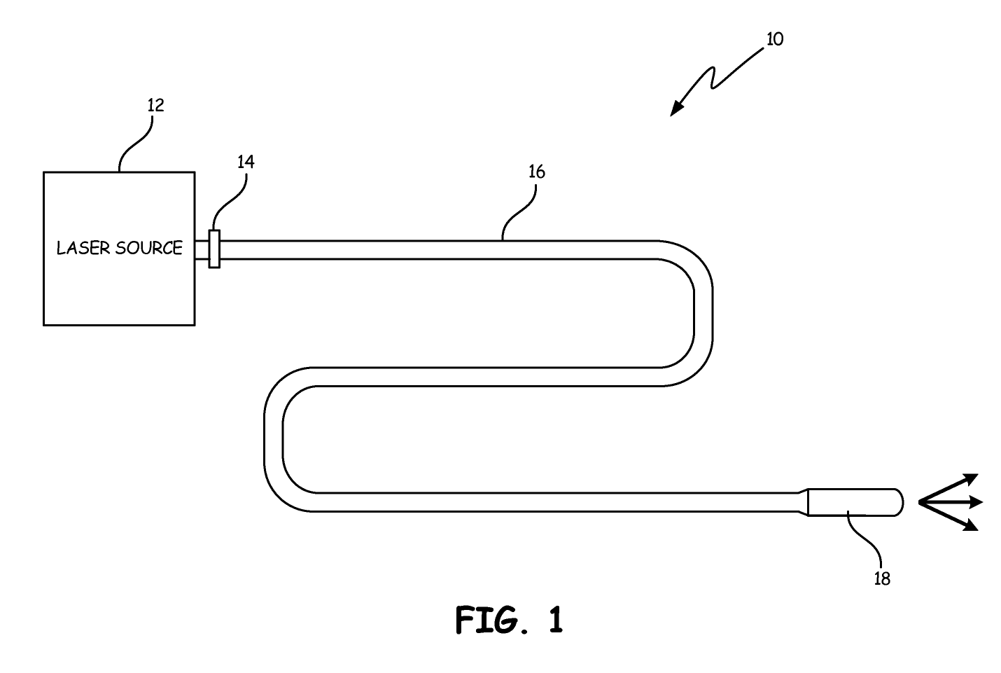

FIG. 1 is a schematic representation of a forward-firing optical fiber system 10 according to an embodiment of the disclosure. The optical fiber forward-firing system 10 can include a laser source 12, an optical coupler 14, an optical fiber 16, and a forward firing distal end 18. The laser source 12 can include at least one laser that can be used for generating laser energy for surgical procedures. In some embodiments, the laser source 12 includes a Ho:YAG laser, a neodymium-doped:YAG (Nd:YAG) laser, a semiconductor laser diode, or a potassium-titanyl phosphate crystal (KTP) laser. In some embodiments, more than one laser is included in the laser source 12 and more than one laser is used during a surgical procedure. The laser source 12 can also have a processor that provides timing, wavelength, and/or power control of the laser. For example, the laser source 12 can include mechanisms for laser selection, filtering, temperature compensation, and/or Q-switching operations.

The optical fiber 16 can be coupled to the laser source 12 through the optical coupler 14. The optical coupler 14 can be an SMA connector (e.g., SMA-905), for example. The laser connecting element of the optical fiber 16 can be configured to receive laser energy from the laser source 12 and the distal end of the optical fiber 16 can be configured to output the laser energy through the forward firing distal end 18. The optical fiber 16 can include, for example, a core, one or more cladding layers about the core, a buffer layer about the cladding, and a jacket. The core can be made of a suitable material for the transmission of laser energy from the laser source 12. In some embodiments, when surgical procedures use wavelengths ranging from about 450 nm to about 2200 nm, the core can be made of silica with a low hydroxyl (OH⁻) ion residual concentration. An example of using low-hydroxyl (low-OH) fibers in medical devices is described in U.S. Pat. No. 7,169,140, which is incorporated herein by reference in its entirety. The core can be multi-mode and can have a step or graded index profile.

The cladding can be a single or a double cladding that can be made of a hard polymer or silica. In some embodiments, the one or more layers of the cladding are doped, for example with fluoride. In some embodiments, the one or more silica cladding layers are fused or otherwise bonded to the core.

The buffer can be made of a hard polymer such as Tefzel®, for example. When the optical fiber includes a jacket, the jacket can be made of Tefzel®, for example, or can be made of other polymers.

In one exemplary embodiment, the optical fiber 16 comprises an 800 μm diameter silica core, an 840 μm diameter silica cladding, a 870 μm hard polymer secondary cladding, and a 1,040 μm Tefzel® buffer coating.

The forward firing distal end 18 can include one or more members, elements, or components that can individually or collectively operate to transmit laser energy in centered on a longitudinal axis or centerline of the distal end of the optical fiber core. In an embodiment, the forward firing distal end 18 can have a protective low-profile cover that includes a coating made of a light-sensitive material. In addition, as will be described in more detail below, in some embodiments, the forward firing distal end 18 includes a clear capillary tube disposed over the bare distal end of the optical fiber.

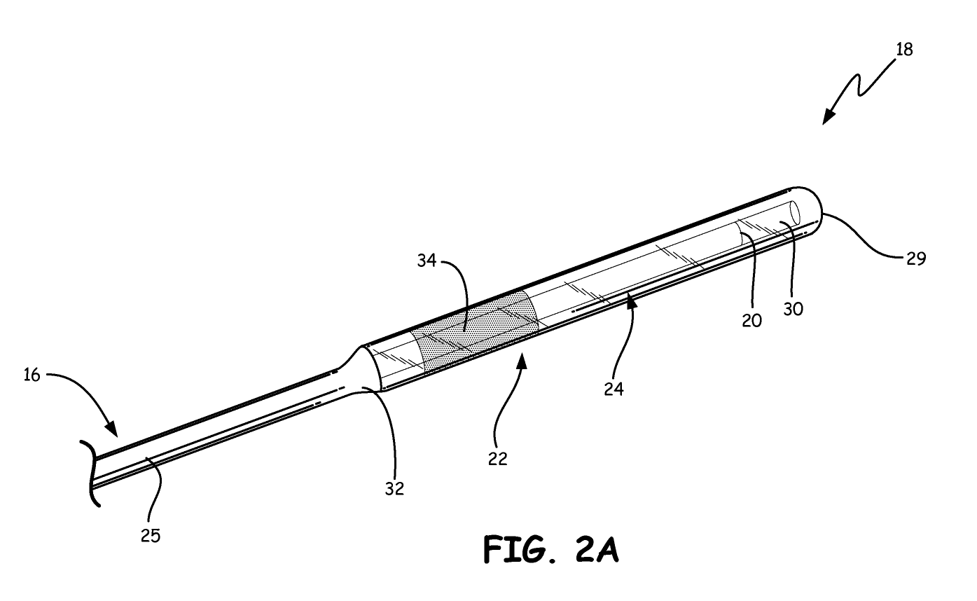

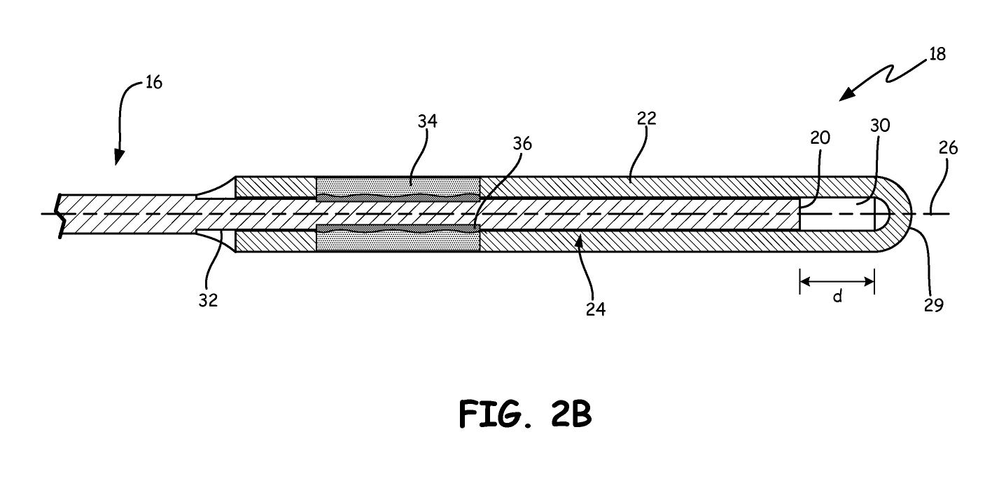

FIG. 2A is a side view and FIG. 2B is a cross-sectional view of an optical fiber forward firing distal end 18 with a flat end face surface 20 disposed within a capillary tube 22, according to embodiments of the present disclosure. In some embodiments, the optical fiber distal end portion 24 includes silica core and cladding layers that are fused or otherwise connected together. The silica core and cladding layers can be exposed at the distal end portion 24 by removing the buffer layer 25 and any other layers surrounding the core and cladding layers 24. A region can be defined within the capillary tube 22 that is configured to receive the forward firing distal end 18. In some embodiments, the capillary tube 22 is approximately 1.0-4.0 cm in length and approximately 2,000 μm in diameter. The optical fiber assembly may be delivered to a treatment site using a cannula that has an inner diameter of about 2,000 μm to allow passage of the capillary tube 22 through the cannula.

As shown in FIGS. 2A and 2B, the optical fiber end portion 24 can include a flat end face surface 20 that is substantially perpendicular to a longitudinal axis or centerline 26 of the optical fiber end portion 24. The flat end face surface 20 can be polished such that the appropriate flatness is achieved. The flat end face surface 20 can be configured such that the flat surface transmits laser energy through the end portion 24 to forward-fire the laser energy through the flat end face surface 20.

In some embodiments, the capillary tube 22 has a rounded or convex distal tip 29. In some embodiments, the capillary tube 22 is comprised of high grade silica. The flat end face surface 20 may be spaced a distance d from the distal end of the interior of the capillary tube 22. In some embodiments, the distance d may be in the range of about 1-4 mm. For example, in one exemplary embodiment, the distance d is about 2 mm. The space 30 in the capillary tube 22 and the distal end of the interior of the capillary tube 22 may be filled with air.

Fluence is diluted at the forward firing flat end face 20 by having the laser energy 28 pass from the fiber end face 20, through air, then through the convex shaped tip 29 of the cylindrical capillary tube 22 where it then radiates tissue. This avoids high microthermal temperatures at the laser discharge from the flat end face surface 20, where the beam diameter is narrowest and fluence or energy density is the highest. For example, in fiber-delivered surgical procedures in which the fiber tip makes direct contact with targeted tissues and photothermal effects are desired, but photoablative effects are determined to be deleterious, the optical fiber assembly of the present disclosure minimizes the photoablative potential, while also minimizing the photothermal density of laser energy at the interface between the outermost capillary tube convex tip and target tissue.

Additionally, the forward firing end face 20 of the fiber 14 is encased and hermetically sealed within the capillary tube 22 to keep the fiber end face 20 clean, dry, and free of contamination, carbonization, or direct exposure to tissue.

Further, tissue trauma is minimized during surgery (e.g., laser lipolysis), since the fiber, which may have a sharp edge around the polished or cleaved end face 20, is encased within the capillary tube 22 having a bullnose or blunt ended convex-shaped tip 29. As a result, the patient experiences less intraoperative and postoperative pain, bruising, bleeding, and swelling, along with an increased safeguard against unintended perforation of tissues beneath or surrounding the target site.

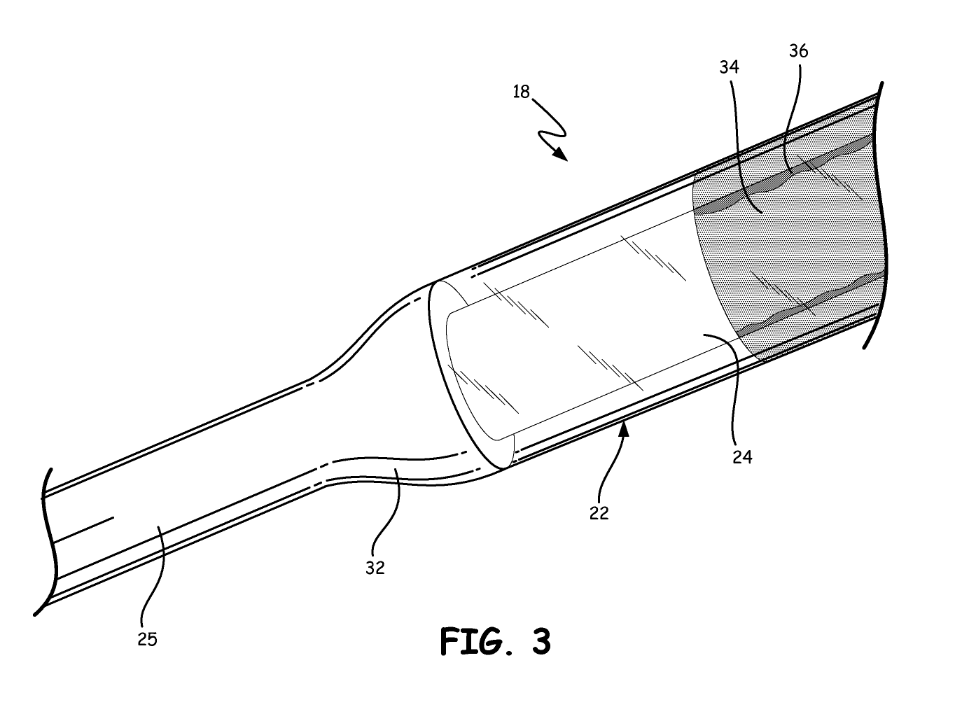

FIG. 3 is a side view of the optical fiber 16, illustrating in more detail the transition region 32 between the capillary tube 22 and buffer layer 23, and the one more fusion regions 34 between the capillary tube 22 and the optical fiber end portion 24. In some embodiments, a proximal end portion of the capillary tube 22 can be coupled to a distal end portion of a buffer layer 23, and/or jacket (not shown) of the optical fiber 16 with a heat stable epoxy. In some embodiments, the capillary tube 22 has an outer diameter larger than the outer diameter of the buffer layer 23 of the optical fiber 16. In such embodiments, the outer diameter in the transition region 32 transitions in size from the buffer layer 23 to the capillary tube 22 to minimize edges or other structures on the optical fiber 16 that could impede the delivery of the forward firing distal end 18 to the tissue to be treated. For example, the transition may be effected with a heat stable epoxy or other formable substance between the buffer layer 23 and capillary tube 22. In some embodiments, the heat stable epoxy provides a substantially edgeless transition region 32 between the buffer layer 23 and capillary tube 22.

One or more fusion regions 34 connect the capillary tube 22 to the optical fiber end portion 24. While one fusion region 34 is shown in FIGS. 2A, 2B, and 3, multiple fusion regions 34 can be formed between the capillary tube 22 and optical fiber end portion 24. The one or more fusion regions 34 are formed between the proximal end of the capillary tube and the end face 20. The one or more fusion regions 34 comprise a melt 36 (see FIGS. 2B and 3) between the capillary tube 22 and the silica cladding and core layers of the optical fiber end portion 24. In some embodiments, the one or more fusion regions 34 comprise circumferential fuses between the capillary tube 22 and optical fiber end portion 24. The one or more fusion regions can be any length, and can be continuous or interrupted along the length of the optical fiber end portion 24 between the proximal end of the capillary tube and the end face 20. In some embodiments, a CO₂ laser is used during manufacturing to form the fusion regions 34.

In some embodiments, to minimize laser energy reflections that can occur between the optical fiber 16 and the capillary tube 22, the refractive indices of the buffer layer 23 and/or the cladding layer of the optical fiber 16 can be substantially matched to the refractive index of the capillary tube 22. Reducing or minimizing the formation of bubbles, air gaps, and/or defects at the one or more fusion regions 34 during the fusion process can also minimize interface reflections. The cladding and/or buffer layer OH⁻ ion concentration can also be controlled to match that of the capillary tube 22. Matching refractive indices can improve the mechanical and/or optical integrity of the one or more fusion regions 34 by minimizing thermal behavior differences between the distal end portion of the optical fiber 16 and the capillary tube 22.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the above described features.

I claim:

1. A forward firing optical fiber assembly comprising:
an optical fiber including a substantially flat end face at a distal end of the optical fiber, the end face extending in a plane substantially perpendicular to a longitudinal axis of the optical fiber; and
a capillary tube including a rounded distal tip, the distal end of the optical fiber disposed within the capillary tube such that the end face of the optical fiber is disposed a distance from an interior distal end of the capillary tube, wherein the forward firing optical fiber assembly includes one or more fusion regions between the optical fiber and capillary tube, the one or more fusion regions configured to hermetically seal the end face within the capillary tube.

2. The optical fiber assembly of claim 1, wherein the fusion region comprises a melt between one or more cladding layers of the optical fiber and the capillary tube.

3. The optical fiber assembly of claim 1, wherein a buffer layer of the optical fiber has a first outer diameter, the capillary tube has a second outer diameter greater than the first outer diameter, and a diameter of a transition region between the buffer layer and capillary tube transitions from the first diameter to the second diameter.

4. The optical fiber assembly of claim 3, wherein the transition region comprises a heat stable epoxy.

5. The optical fiber assembly of claim 1, wherein a space within the capillary tube between the end face and an interior distal end of the capillary tube comprises air.

6. The optical fiber assembly of claim 1, wherein the distance is less than about 2 mm.

7. The optical fiber assembly of claim 1, wherein a length of the capillary tube is approximately 1-4 cm.

8. A surgical laser system comprising:
a laser source; and
an optical fiber assembly optically coupled to the laser source, the optical fiber assembly comprising an optical fiber including a substantially flat end face at a distal end of the optical fiber, the end face extending in a plane substantially perpendicular to a longitudinal axis of the optical fiber, the optical fiber assembly further including a capillary tube including a rounded distal tip, the distal end of the optical fiber being disposed within the capillary tube such that the end face of the optical fiber is disposed a distance from an interior distal end of the capillary tube, wherein the forward firing optical fiber assembly includes one or more fusion regions between the optical fiber and capillary tube configured to hermetically seal the end face within the capillary tube, and
wherein the optical fiber assembly is configured such that laser energy provided by the laser source to the optical fiber assembly is transmitted through the end face along the longitudinal axis of the optical fiber through a distal end of the capillary tube.

9. The surgical laser system of claim 8, wherein the fusion region comprises a melt between one or more cladding layers of the optical fiber and the capillary tube.

10. The surgical laser system of claim 8, wherein a buffer layer of the optical fiber has a first outer diameter, the capillary tube has a second outer diameter greater than the first outer diameter, and a diameter of a transition region between the buffer layer and capillary tube transitions from the first diameter to the second diameter.

11. The surgical laser system of claim 10, wherein the transition region comprises a heat stable epoxy.

12. The surgical laser system of claim 8, wherein a space within the capillary tube between the end face and an interior distal end of the capillary tube comprises air.

13. The surgical laser system of claim 8, wherein the distance is less than about 2 mm.

14. The surgical laser system of claim 8, wherein a length of the capillary tube is approximately 1-4 cm.

15. A forward firing optical fiber assembly comprising:
an optical fiber including a buffer layer, one or more cladding layers, and a core, at least one of the cladding layers coupled to the core, wherein the buffer layer is removed at a distal end portion of the optical fiber to expose the coupled at least one cladding layer and core, wherein the distal end portion includes a substantially flat distal end face, the end face extending in a plane substantially perpendicular to a longitudinal axis of the optical fiber;
a capillary tube including a rounded distal tip, the distal end of the optical fiber disposed within the capillary tube such that the end face of the optical fiber is disposed a distance from an interior distal end of the capillary tube; and
one or more fusion regions between the distal end portion and capillary tube, the one or more fusion regions configured to hermetically seal the end face within the capillary tube.

16. The optical fiber assembly of claim 15, wherein the fusion region comprises a melt between the distal end portion of the optical fiber and the capillary tube.

17. The optical fiber assembly of claim 15, wherein a buffer layer of the optical fiber has a first outer diameter, the capillary tube has a second outer diameter greater than the first outer diameter, and a diameter of a transition region between the buffer layer and capillary tube transitions from the first diameter to the second diameter.

18. The optical fiber assembly of claim 17, wherein the transition region comprises a heat stable epoxy.

19. The optical fiber assembly of claim 15, wherein a space within the capillary tube between the end face and an interior distal end of the capillary tube comprises air.

20. The optical fiber assembly of claim 15, wherein the distance is less than about 2 mm.
